(19) United States
(12) Patent Application Publication
(10) Pub. No.: US 2016/0121017 A1
(43) Pub. Date: May 5, 2016

(54) SINGLE SOLUTION of Gel-LIKE FIBRIN HEMOSTAT

(71) Applicant: George Falus, New York City, NY (US)

(72) Inventor: George Falus, New York City, NY (US)

(73) Assignee: Biomedica Management Corp, Baltimore, MD (US)

(21) Appl. No.: 14/532,514

(22) Filed: Nov. 4, 2014

(51) Int. Cl. A61L 24/04; A61L 24/00

(52) U.S. Cl. CPC A61L 24/043; A61L 24/0015; A61L 24/0031; A61L 2400/04; A61L 2300/418

(57) ABSTRACT

The present invention trademarked ClotGel© is a fibrin II-based hemostat made of two components that are mixed into a single syringe to be delivered as an adjunct or primary treatment in moderate intraoperative hemorrhage and in trauma. It can be applied topically to the wound either on the skin in a laparatomy or as non-invasive manner in surgical procedures. Its cross-linking technology generates an adhesive stable fibrin clot required for hemostasis. The agent consists of a cross-linked gelatin that is homogenized in a solution of fibrin monomer in acetic acid, which is reconstituted before use from a lyophilized fibrin monomer. When both components are mixed into a syringe they produce a viscous gel-like composition that is polymerized and stabilized when in contact with blood. The attachment properties of the composition, as well as the rapid formation of a fibrin clot, ensures that a strong stable blood clot is formed over a bleeding wound within 2 minutes of application.

### RELATED APPLICATIONS

This application is a Continuation-In-Part of U.S. patent application Ser. No. 13/731,120 confirmation number 1007. All description, drawings and teachings set forth therein are expressly incorporated by reference herein and I claim priority upon the teachings expressly made therein.

### FIELD OF THE INVENTION

The present invention is related to a fluid desAB fibrin (fibrin II) monomer gel-like composition, and a method to produce this composition or “aquagel” from desAB fibrin (fibrin II) monomer in acid solution to seal tissue, and for use as a hemostatic agent to control vascular, epidermal, bone, or internal hemorrhage. The invention under trade name ClotGEL© is a fibrin II monomer-based solution into which a dry-cross-linked gelatin is homogenized to form of a fluid viscous gel-like solution that may be used as an hemostat to stop bleeding or seal tissue in vivo with and without compression. It is particularly related to a method that allows reconstituting into a solution a lyophilized fibrin II monomer to form a fluid viscous gel-like composition when cross-linked gelatin is homogenized into this solution. The fluid viscosity of the gel-like composition is calculated to facilitate flow through the syringe while resisting the flow of blood from a wound without need of a biodegradable support. The resulting mixture of cross-linked gelatin and fibrin monomer solution has been found to be a highly effective hemostatic sealant when applied to a bleeding site.

The present invention lies within the domain of biological tissue sealants and hemostats, which are biodegradable and nontoxic, intended for therapeutic use, for example, as an adjunct to hemostasis in laparotomy or laparoscopic surgery, in orthopedic surgery, trauma (spleen laceration), and large-bed wounds, burns, or as a primary treatment in biopsies, spleen lacerations, etc.

### BACKGROUND OF THE INVENTION

Moderate bleeding for organ resection, some types of trauma, bone fractures, orthopedic procedures, vascular anastomosis, solid organ wounds, and deep dermal wounds often require an adjunct to control the bleeding before or after sutures or stitches are applied. Also burns and some traumatic events such as spleen laceration that cannot be sutured, or bleeding from biopsies that could be controlled with no need of sutures or staples, require a primary treatment that can stop the bleeding.

Various technologies have been developed for the formulation of hemostats. Some of them contain biological materials such as collagen or fibrinogen and thrombin. As a result of their hemostatic and adhesive properties, fibrin sealants, or thrombin-based products have been extensively used in most surgical specialties for over two decades to reduce blood loss and post-operative bleeding due to their ability to adhere to human tissue as it polymerizes (1, 2, 3). These compounds are used to seal wounds that have been sutured or stapled; they can also be used with pressure over an injured area to control bleeding. Fibrin sealants are biological adhesives that mimic the final step of the coagulation cascade. (4) The main components of current sealants in the market are fibrinogen and factor XIII on the one hand, and thrombin and calcium chloride on the other. The components are often extracted from human plasma or produced by recombinant techniques. Cleaving fibrinogen with thrombin creates a polymer barrier (fibrin II) that simulates the last stages of the natural coagulation cascade to form a structured fibrin clot similar to a physiological clot. Products containing thrombin stimulates the coagulation cascade forming a natural fibrin clot.

There are several commercial products available (Floseal, Gelfoam, Evicel, Bioglue) (5). However, these products have limitations such as inflammatory effects, immunological responses, frozen storage conditions, and cumbersome preparation. Most hemostatic agents for intracavitary bleeding are designed to be used in case of light to moderate bleeding in the operating room, and require hard compression. One of the major limitations encountered in the development and/or use of fibrin compositions made of fibrinogen and thrombin with brief or no compression is their inability to form an instant polymer that resists the flow of blood, a strong bond to tissues, a stable clot within minutes of application, and in addition, require to incorporate thrombin and anti-fibrinolytic agents in the formulation with potentially adverse events. However, there are many situations where the use of strong compression, sutures and/or staples is undesirable, inappropriate or impossible, (e.g. in bone, brain, interventional radiology, retroperitoneal surgery). Also the inclusion of thrombin prevents the recover of lost blood. And finally fibrin-based hemostats require to be stored frozen (−18° C.) to maintain stability, which substantially increases the shipping cost, and require a time consuming preparation before surgery.

The present invention overcomes most of the limitations encountered in flowable hemostats by eliminating the presence of thrombin in the composition applied to the wound, storing the composition at room temperature, producing a flowable hemostats that resist the flow of blood, minimizing the time of preparation before use, extending the shelve life, reducing the amount of fibrin need to achieve rapid hemostasis, and consequently the cost of manufacturing. These objectives are fully met in the invention described hereinafter.

Converting fibrin monomer solution in acetic acid into a dry stable powder that could be rapidly reconstituted without loss of activity or molecular degradation was essential to storing the fibrin sealant at room temperature. Although lyophilization of fibrinogen into rapidly soluble microparticles has been achieved (US patent 2010/0150900 and U.S. Pat. No. 6,113,948), the conversion of a fibrin II monomer solution into a dry powder, which presents major challenges due to the termostability of the protein and the predisposition of the fibrin monomer to polymerize, has never been achieved before, nor can be inferred by persons experienced in the art. Stabilization during lyophilization of proteins has previously focused on the use stabilizers as well as well as the dehydration process method and parameters. However, the effect of protein crystallization and the reconstruction medium has largely been neglected. Our preliminary melting experiments performed using Circular Dichroism (CD) method, indicate that addition of sucrose to fibrin monomer solution may significantly stabilize its structure shifting the beginning of its denaturation (melting) from 58 to 74° C. (FIG. 1). Taking also into account previous studies on the use of saccharides to maintain stability of plasmatic proteins (7) through a dry spray Lyophilization process, we developed a novel method to convert the fibrin II monomer solution into a dry powder with a very rapid reconstitution—less than two minutes—and high yield recovery of 85% the protein with absence of agglomerates. Furthermore, our preliminary data indicate that treatment of fibrin monomer in such manner allows recovering more that 85% of functionally active protein.

The use of gelatin to make a viscous gel-like solution is well established in the food and pharmaceutical industry. Although porcine or bovine gelatin does not interfere in the polymerization process of fibrin monomer in solution, and it is effective in producing a gel-like fluid that can resist the flow of blood, when gelatin is mixed with fibrin monomer solution the homogenized composition tends to form a non-fluid gel or large agglomerates due to a tri-dimensional cross-linked network with the fibrin monomer solution. This agglomeration limits the flowability of the composition to 10 minutes, at which point in time the composition cannot be anymore extruded through the syringe. A glutaraldehyde cross-linked gelatin homogenized into the fibrin monomer solution prevents this agglomeration maintaining a more stable fluid property of the homogenized fibrin monomer composition for use within one hour of preparation.

The ClotGel product is presented in a package that includes three components: 1) a syringe filled with dry cross-linked gelatin; 2) a syringe filled with microparticles of lyophilized fibrin monomer; and 3) a syringe filled with acetic acid pH 3.5 for reconstitution of the lyophilized fibrin monomer. (FIG. 5). Immediately prior to use in the operating room, the lyophilized fibrin monomer is reconstituted and the cross-linked gelatin is homogenized into the fibrin monomer solution in less than three minutes. The hemostat is then ready to be applied within one hour of preparation through the syringe in which composition has been homogenized. The fibrin monomer is neutralized by the flowing blood which raises the pH to 7.0, consequently forming a fibrin clot almost instantly. Factor XIII present in the blood cross-links the fibrin clot within 2 minutes of application.

### Background Art

The preparation of fibrin monomer in acetic acid solution is described in U.S. Pat. No. 8,367,802 by G Falus et al. The use of trehalose and or other saccharides for raising the termoestability point of fibrin are described in Effect of trehalose on protein structure Nishant Kumar Jain and Ipsita Roy, in US 20100150900 A1, and Hemostatic Properties of Infusible Trehalose-Stabilized Lyophilized Platelet Derivatives. Keith A. Moskowitz, Josh Dee, Jason Barnidge, Ruth Sum, David Ho, Alan S. Rudolph, and Cindy S. Orser, among other publications.

A two-step process of freeze dry spaying to lyophilize fibrin monomer is described in Improving Powders with Freeze Granulation, Ceramic Industry, Apr. 40-44, 2003; Si₃N₄ Powders Applied for Water-Based DCT, Ceramic Transaction 142, 44-62, 2003; Development of Water-Based Processing of Silicon Nitride Materials, Ceram. Eng. Sci. Proc., 23 (3) 3-10, 2002; The Influence of the Granule Structure on the Strength of Pressed and Sintered Si₃N₄, Sixth ECerS; Freeze-Granulation of Liquid Phase Sintered Silicon Carbide, Ceramic Transactions 42, 1994; Granulation of Ceramic Powders for Pressing by Spray-Freezing and Freeze-Drying, Euro-Ceramics II, Vol. 1, 1993; Powder formation by atmospheric spray-freeze drying U.S. Pat. No. 8,322,046 B2

A method to crosslink gelatin with glutaraldehyde as to obtain a stable biocompatible dry gelatin is described in U.S. Pat. No. 7,547,4461; J Biomater Appl October 1999 vol. 14 no. 2 184-191 Swelling Behavior and Mechanical Properties of Chemically Cross-Linked Gelatin Gels for Biomedical Use; Protein Sci. Jan 2009; 18(1): 24-36, among other publications

### SUMMARY OF THE INVENTION

In the first aspect, the present invention relates to biocompatible adhesive fibrin polymer, which is bio-reabsorbable and nontoxic, for surgical or therapeutic use. It also relates to a single-part homogenized composition containing a single bioactive substance (fibrin II monomer) which can be released in a given site to produce a stable fibrin clot, and form a physical barrier that resists the flow of blood. The adhesive matrix of fibrin-based hemostat must form in a matter of seconds a strong fibrin interface, bond with tissues in the midst of flowing blood and remain at the lacerated site to form a clot. U.S. Pat. No. 8,367,802 by Falus et al. describes a method of preparing a fibrin monomer. Unlike other fibrin sealants that use thrombin to cleave fibrinogen in situ to form a desAB fibrin II (fibrin II) polymer, the invention polymerizes desAB fibrin monomer by neutralization in contact with the blood to produce a fibrin clot, thus bypassing the cleavage process. The essential aspect of the technology is the ability of desAB fibrin (fibrin II) monomer to be mixed with the blood to form a physical barrier that turns into a functional fibrin clot.(8) In our approach these results are obtained through the in-situ generation of a polymeric cross-linked fibrin network that is bonded to the tissue as a strong fibrin clot stabilized by factor XIII present in the blood, and optionally by tranglutaminase enzyme ACTIVA© added to the composition.

Hemostasis is achieved as a result of pH neutralization of the fibrin monomer present in the composition when in contact with the wound blood, turning the composition into a polymer of long fibers, and trapping the blood cells. The transglutaminase enzyme in the form of ACTIVA, that can optionally be added to the composition, and activated factor XIII present in the blood leads to further formation of covalent bonds in the clot. The inclusion of calcium-independent transglutaminase enzyme facilitates the transglutaminase-mediated oligomerization of the αC-domains of fibrin promoting integrin clustering and thereby increasing cell adhesion and spreading, which stimulates fibrin to bind αvβ3-, αv-β5- and α5-β1-integrins on endothelial cells (9). The oligomerization also promotes integrin-dependent cell signaling via focal adhesion kinase (FAK) and extracellular signal-regulated kinase (ERK), which results in an increased cell adhesion and cell migration[10]. The clot is mechanically stable, well integrated into the wound and more resistant to lysis by plasmin compared with uncrosslinked clots [8] or other fibrin sealants.

In a second aspect, the invention relates to a process for producing a fluid viscous composition that can resist the flow of blood. The ability of this composition to adhere to human tissue is related to the use of a cross-linked gelatin, which is homogenized in desAB fibrin monomer or fibrin II solution in acetic acid. A gelatin cross-linked by glutaraldehyde following a method described in this invention maintains relatively constant the viscosity, preventing cross-linking with the monomer solution, and thus preserving the flowability of the homogenized composition without interfering with the fibrin monomer polymerization process.

In third aspect, the invention relates to the lyophilization of fibrin II monomer solution in a way that allows for rapid reconstitution instead of freezing the fibrin solution for preservation, in order to reduce preparation time before surgery, as well as shipping and storage costs. The fibrin monomer II incorporated in this composition is in a lyophilized form and ready to be reconstituted in less than two minutes without loss of efficacy and molecular integrity.

The ClotGel sealant composition uses a two-step process for lyophilizing fibrin II monomer prepared with dihydraded trehalose which is reconstituted in acetic acid and polymerized when in contact with blood. The composition and method of production of the fibrin monomer (e.g. concentration of thrombin and fibrinogen to produce a fibrin II polymer, and dissolution in acetic acid by dialysis) described in U.S. Pat. No. 8,367,802 by G Falus et al, herein incorporated by reference, has an important effect on the type of polymer, once the monomer is reconstituted, because the performance of the proposed technology depends on the characteristics of fibrin itself (Clotting time, clottability, thickness of the fibers, the number of branch points, the porosity, and the permeability). The blood clot produced by the reconstituted fibrin monomer in this invention creates opaque matrices of thick fibers, and, therefore, formation proceeds at a faster rate than in transparent matrices. The two-step lyophilization process of fibrin monomer solution consisting of a first freeze-spray in liquid nitrogen, and second a freeze-dry in a tray style lyophilizer produces a powder that, unlike the standard one step freeze dry or dry spray, allows for reconstitution of fibrin in less than two minutes instead of 20-30 minutes with no agglomerates and loss of material.

In a fourth aspect of the invention relates to a novel fibrin sealant composition that incorporates lyophilized fibrin II monomer, that once reconstituted with acetic acid pH 3.5 into a fibrin monomer solution, is homogenized in a glutaraldehyde cross-linked porcine gelatin. The homogenized gelatin-monomer mixture is ready to polymerize at change of pH when in contact with the blood. Mixing blood with the gel-like or aquagel composition creates a mesh of fibrin fibers that form the fibrin clot at sites of injury (11). Under coagulant conditions, optional inclusion in the formulation of calcium-independent transglutaminase enzyme and activated Factor XIII present in the blood contribute to this process by stabilizing the fibrin clot through covalent bonds.

In a fifth aspect, of the invention provides a syringe container system to mix and apply the components.

In a sixth aspect, the invention provides the use of the composition for the hemostatic control of bleeding, and as fibrin sealant.

In a seventh aspect, the invention allows the use of final sterilization by gamma radiation.

The invention thus provides a hemostatic agent, which has been tested in vitro and in vivo. The data obtained presents ample evidence of the ability of ClotGel to stop bleeding and achieve hemostasis with and without compression in three surgical protocols including liver biopsy, partial nephrectomy and spleen laceration in the swine models. The polymerization process begins instantly at the time of application. Extensive in vivo studies have shown that ClotGel is an excellent general hemostat for use as adjunct and primary treatment in moderate bleeding. The agent is durable, easy to store, poses minimal risk, requires little training to use, and is highly effective against bleeding.

### BRIEF DESCRIPTION OF THE DRAWINGS

The various features, advantages and other uses of the present invention will become more apparent by referring to the following detailed description and drawings in which:

FIG. 1 CD-detected melting curves of fibrin monomer at 0.5 mg/ml in acetic acid, pH 3.5 (A), and with added of sucrose, (B).

FIG. 2A Process equipment to freeze spray fibrin monomer in solution in liquid nitrogen

FIG. 2B Process to freeze spray fibrin monomer in solution in liquid nitrogen, and freeze dry the frozen micro-particles

FIG. 3 Magnification of fibrin monomer frozen microparticles of a size below 100 μm

FIG. 4A Agglomeration of non cross-linked gelatin particles following homogenization in fibrin monomer solution

FIG. 4B Agglomeration of cross-linked gelatin particles following homogenization in fibrin monomer solution

FIG. 5 Set of syringes to store the components of the composition

FIG. 6 Male luer-lock syringe attached to female luer-lock syringe used to homogenize the gelatin into the fibrin monomer solution

FIG. 7 Rate of reconstitution (percentage of powder dissolved in seconds) of fibrin monomer/trehalose microparticles compared to pure fibrin monomer microparticles, and fibrin monomer lyophilized directly in a freeze dry system.

FIG. 8 Clotting time of reconstituted lyophilized fibrin monomer at concentrations ranging from 6 mg/mL as compared to non-lyophilized fibrin monomer

FIG. 9 Turbidity assay comparing rates of polymerization at pH 3.5 and pH 4.5 between non-lyophilized and lyophilized fibrin monomer

FIG. 10 Analysis of the sedimentation profiles by ultracentrifugation of three samples, Fibrinogen used as raw material for production of fibrin monomer, Fibrin from non-lyophilized material and lyophilized fibrin monomer

FIG. 11 HPLC Size exclusion chromatography (SEC) lyophilization process comparing non-lyophilized monomer with lyophilized fibrin monomer mixed with trehalose,

FIG. 12 SDS-page analysis of polymerization and cross-linking of lyophilized fibrin monomer and non-lyophilized fibrin monomer in the presence of factor XIII and ACTIVA as cross-linking agents

FIG. 13 A Scanning electron microscopy of fibrin fibers produced by non-lyophilized

FIG. 13 B Scanning electron microscopy of fibrin fibers produced by lyophilized fibers

FIG. 14 Western Blot analysis of non-lyophilized and lyophilized fibrin(ogen)s and their cross-linking by activated FXIII and/or Activa at pH 3.5 and 4.0.

FIG. 15 Average Clotting Time of lyophilized fibrin monomer stored at 27° C. for 60 days and projected values for 360 days

FIG. 16 Average Clotting Time of non-lyophilized fibrin monomer stored at −20° C. for 308 days and reconstituted.

FIG. 17 Clottability of reconstituted lyophilized fibrin monomer stored at 27° C. for 50 days as compared to non-lyophilized monomer and non-lyophilized monomer with preservatives.

FIG. 18 Cross-linking at 5 minutes following neutralization of fibrin monomer preserved at 27° C. and reconstituted at time periods between 0 and 60 days

FIG. 19 A Stability of lyophilized fibrin monomer stored at 27° C. for 0 days measured by SDS page

FIG. 19 B Stability of lyophilized fibrin monomer stored at 27° C. for 90 days measured by SDS page

FIG. 20 Polymerization, cross-linking and stabilization of reconstituted lyophilized fibrin monomer in the presence of ACTIVA, Factor XIII and Activa+factor XIII

FIG. 21 Cross-linking of porcine gelatin by glutaraldehyde dissolved in ethanol and water and in water alone.

FIG. 22 A Biocompatibility experiments performed in human fibroblast (HF) cultures exposed to ClotGel preparations. (HF−Untreated, Day 5

FIG. 22 B Biocompatibility experiments performed in human fibroblast (HF) cultures exposed to ClotGel preparations. HF+Clotgel, Day 5

FIG. 23 A Biocompatibility experiments performed in human epithelial cell cultures (A549) exposed to ClotGel preparations. A549 cells−Untreated, Day 5

FIG. 23 B Biocompatibility experiments performed in human epithelial cell cultures (A549) exposed to ClotGel preparations. A549 cells+ClotGel, Day 5

FIG. 24. ClotGel applied over a lacerated surface

### DETAILED DESCRIPTION

The present invention consists of a hemostatic composition and tissue sealant comprising:
a) A dry stable chemical active entity consisting of a lyophilized fibrin II monomer,
b) a dry cross-linked gelatin,
c) acetic acid at pH 3.5
d) A set of syringes to reconstitute the lyophilized fibrin monomer and homogenize the gelatin with the fibrin monomer solution into a single gel-like composition that is applied over a bleeding site.

This formulation can be activated by reconstituting the lyophilized monomer in acetic acid solution at pH 3.5, and subsequently homogenizing it with cross-linked gelatin that acts as a viscosity agent, to form a fluid aquagel for use as an adjunct to hemostasis in general surgery or primary treatment in surgical procedures, and in the treatment of traumatic wounds, to create hemostasis through the formation of a stable fibrin clot when in contact with blood. Hemostasis can be achieved with or without compression. Each component is stored in three separate syringes, which are mixed before usage to produce a single viscous gel-like product.

The invention is a novel fibrin sealant depleted of thrombin and without any other solid polymer support that offers optimal viscoelastic properties to resist the flow of blood, maintain a stable viscosity and fluid properties within one hour of preparation, and rapidly produce a fibrin clot at the wound site.

The lyophilized fibrin monomer is produced by freeze spraying a solution made of fibrin II monomer mixed with trehalose in liquid nitrogen, and by subsequently freeze drying the frozen particles of fibrin monomer. The concentration of fibrin monomer in the particles is preferably 20%

The fibrin II monomer solution in acetic acid pH 3.5 to be lyophilized is made by cleaving USP human fibrinogen with USP thrombin in the presence of calcium ions, and subsequently incubating the fibrin II polymer, and dissolving it in acetic acid by dialysis into fibrin monomer solution at pH 3.5 according to the proprietary method described in U.S. Pat. No. 8,367,802, Falus et al.

Alternatively, recombinant DNA technology or USP salmon fibrinogen can be used in replacement of human fibrinogen, and recombinant thrombin in replacement of human or bovine thrombin (12).

Fibrin II monomer solution preferably at a concentration of 30 mg/mL to 40 mg/mL is mixed in with and additive material as a stabilizer, preferable dihydrated trehalose, and sprayed through a nozzle into liquid nitrogen (FIG. 2A). The frozen particles of fibrin monomer are subsequently freeze dried for 24 hours in shelf freeze drier apparatus (FIG. 2B).

Microparticles from 10 μm up to 50 μm of lyophilized fibrin monomer may be prepared by methods described above in the background art section. These two-step process of freeze spraying in nitrogen and subsequently freeze drying the frozen particles of fibrin monomer in a tray style lyophilizer enables the production of soluble fibrin monomer that can be reconstituted in acetic acid pH 3.5 within one minute.

The frozen particles of fibrin monomer may be prepared by dissolving a highly soluble saccharide such as dehydrated trehalose in the fibrin monomer solution before freeze spraying the solution. Other biocompatible materials are suitable for use as water absorbent additive that increases the rate of reconstitution of the fibrin monomer powder and matains the stability of the fibrin molecule. Such materials include hyaluronic acid, dextran polymers and starches that may be incorporated into the fibrin monomer solution as single components or in combination before freeze spraying. Typically the composition will include at least 1 gr. of additive material per mL of fibrin monomer solution up to 10 grs of additive material per mL of monomer solution, being a (5:1) of weight in gr of the saccharide per mL of fibrin solution the preferred ratio.

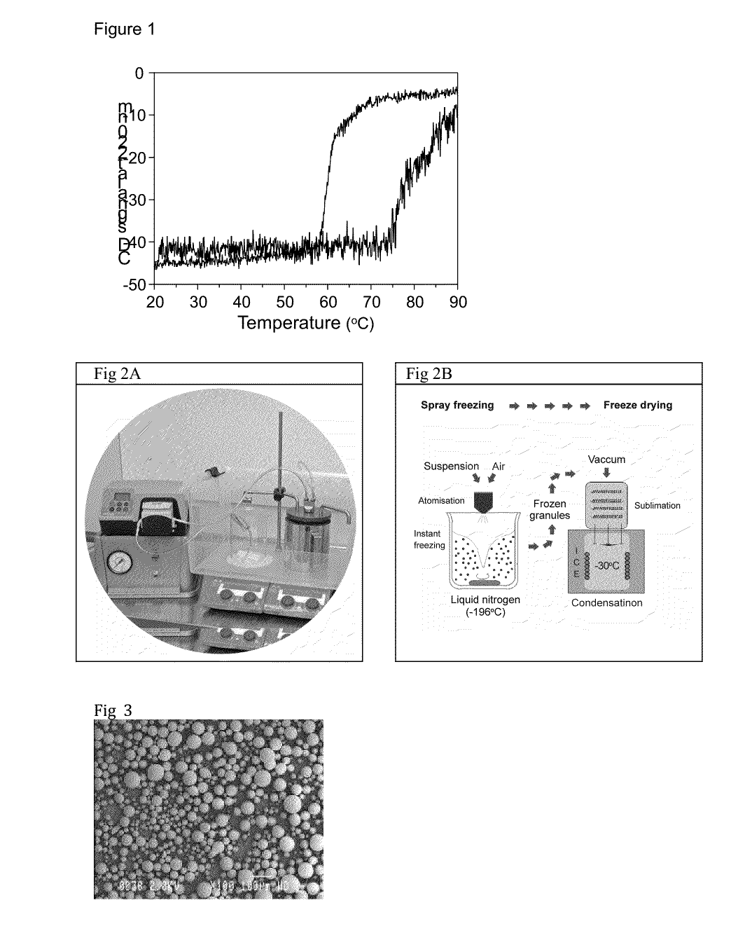

The additive material increases the termostabiltity of the protein up to 75° C. as shown in FIG. 1 that protects its molecular integrity through the Lyophilization and sterilization process enabling the composition to be stored at room temperature or in hot weather, for extended periods of time, for example greater than 6 months.

Frozen sphere microparticles (FIG. 3) of fibrin monomer are prepared in a freeze spray apparatus shown in FIG. 2A, which utilizes compressed air to atomize the particles through a fluid nozzle into a vessel containing liquid nitrogen. Subsequently the frozen particles are dried overnight in tray lyophilizer as shown in FIG. 2B with a residual water content preferably no greater than 5%.

The size of the microparticles can be modified by adjusting the air flow configuration in the freeze drier. When particles agglomerate they can be separate by mechanical means as it would be appreciated by persons skilled in the art. Such agglomerates may be mechanically reduced, being the preferable larger size below 100 μm.

The lyophilized fibrin monomer is filed into a female luer lock syringe of the type Quosina Part C3603 or similar where is stored in powder, and reconstituted before use with acetic acid pH 3.5 to concentration 30 to 40 mg/ml.

A dry powder fibrin monomer that can be reconstituted within one minute preserving its molecular integrity is of important value for the production of fibrin-based sealants by eliminating the need to freeze the protein in order to ship and store it, and eliminates the time-consuming process of thawing it before use in the surgical room.

The fibrin monomer can be sterilized by filtration through a 0.22 micron Millipore filter once in solution, or by gamma radiation at a maximum 30 kGy dose at room temperature once it is stored in powder in the syringe. The radiation dose does not require viral elimination due to the fact that the fibrin monomer is produced from USP fibrinogen and sterility is broken solely by environmental contamination during the manufacturing process.

The glutaraldehyde crosslinked gelatin reduces the formation of cross-linking bonds with the fibrin monomer solution as compared to non-crosslinked gelatin when homogenized in this solution as well as the electroviscous effect (13). Although gelatins can be cross-linked with aldehydes epoxies, carbodiimides, isocyanates and transglutaminase enzymes, among others (11), glutaraldehyde is the preferred method to crosslink the gelatin due the easiness in washing away the glutaraldehyde as well as the agents necessary to stop the cross-linking reaction from the composition. The cross-linked gelatin can be of bovine or porcine origin bloom 300, preferable porcine gelatin type A because it is made by acidic treatment, maintains a higher bloom strength at pH 3.5, and does not modify the pH of the fibrin monomer solution. Before cross-linking the gelatin must be filtered with detoxi-gel (fisher Scientific) to reduce endotoxin content to less 3 units per milliliter.

A cross-linked gelatin by glutaraldehyde is produced by dissolving bovine or porcine gelatin in a water solution, preferably porcine gelatin in a solution of 50% water and 50% ethanol. Preferable the concentration of gelatin in this solution is 10%.

Subsequently, the gelatin solution may be cross-linked by adding 0.1% to 0.2% of glutaraldehyde to the solution and incubating it overnight at room at a temperature of 2° C. if the gelatin solution was prepared with water, or at room temperature if the solution was prepared with water and ethanol.

The cross-linked gelatin obtained in a form of a flurry is filter-washed with a solution 50% ethanol and 50% water to remove the glutaraldehyde. The remaining glutaraldehyde may be inactivated and the cross-linked gelatin stabilized by adding 0.1% of borohydride or 0.1% of glycine in relation to the amount of solution, and incubated at room temperature for 3 hours at pH 8.0-9.0. Filtration washing steps can be repeated one or more times in order to clean the gelatin and remove all residues. The cross-linked gelatin is then filter-washed with 100% ethanol and dried overnight at 37° C. overnight to produce a dry powder

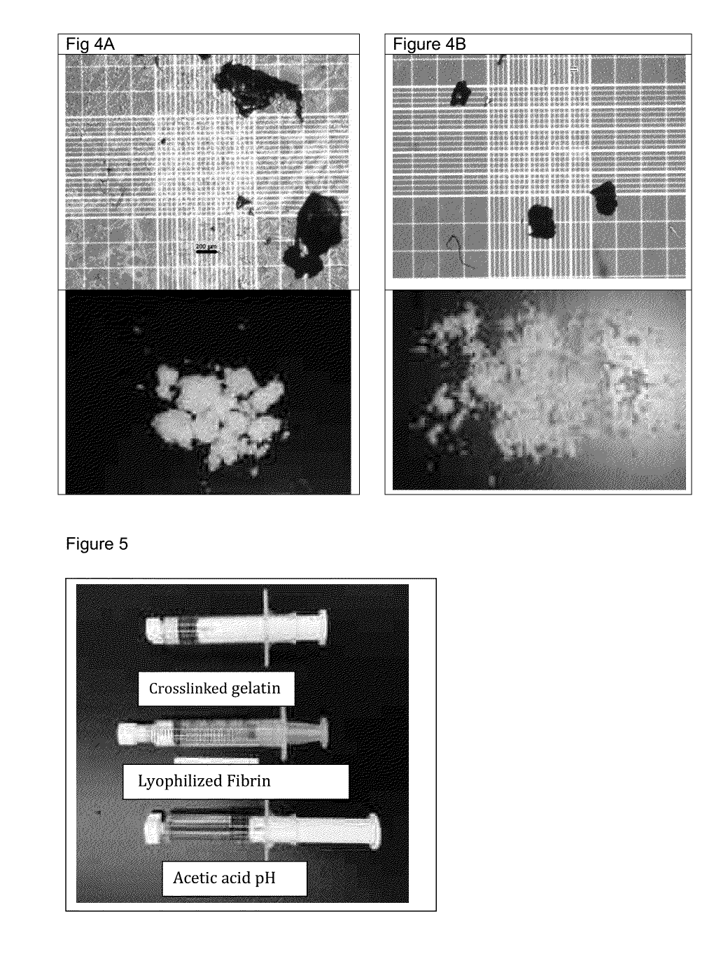

Although the dry cross-linked gelatin powder of present invention has a similar rehydration rate or 1:3 to the non-crosslinked gelatin, the hydrodynamic properties related to the electroviscous effect of solid particles suspended in ionic liquids, and the conformational behavior of the gelatin particles related to its shape and particle interaction differ sharply, preventing the formation of agglomerates. FIG. 4A shows the agglomeration of non-crosslinked gelatin compared to cross-linked gelatin (FIG. 4B), that produces a relatively stable fluid viscous homogenized composition with fibrin monomer. Homogenization with cross-linked gelatin maintains the fluid viscosity from the moment of preparation to one hour time limit between 130,000 and 190,000 cP, and the pressure over a 5 cc syringe piston necessary to dispense the composition, between 2 and 7 PSI

Once dry, the cross-linked gelatin is filed into a female luer lock syringe of the type Quosina Part C3603 or similar and sterilized gamma or e-beam radiation.

Before use, the monomer is reconstituted to 30-40 mg in acetic acid pH 3.5 and mixed (homogenized) into a single syringe. The of amount of the glutaraldehyde-cross-linked gelatin to be homogenized in fibrin monomer solution may be 0.4 grs. to 0.7 grs. for 4 mL of reconstituted fibrin II monomer in acetic acid solution pH 3.5 at a concentration from 12 to 40 mg/ml.

The kit according to the present invention comprises a first female luer lock syringe holding the dry fibrin monomer microparticles as described above; a second male luer locfk syringe containing acetic acid pH3.5; a third female luer lock syringe holding the dry crosslinked gelatin (FIG. 5); and a set of application tips.

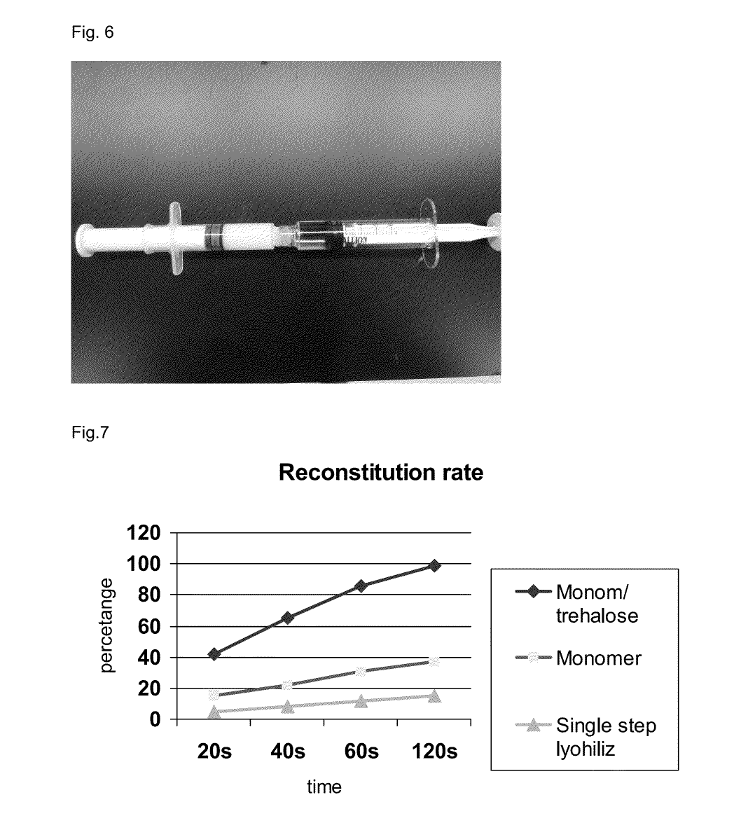

Once the Fibrin monomer is reconstituted the crosslinked gelatin is homogenized into the monomer solution by transferring forth and back the mix from one luer lock syringe to the other (FIG. 6). The gel-like homogenized composition is applied using a syringe with an applicator tip over lacerated bleeding tissue, to form a sticky, gummy barrier that when in contact with blood, rapidly, within 6 seconds, creates a blood Clot. The fibrin clot is biodegraded overtime by fibrinolytic enzymes. The agent binds together with the lacerated tissue, and seals the wound with a stable cross-linked blood clot within 2 minutes of application.

The invention will now be described, by way of illustration, with reference to the following examples

### EXAMPLES

The preparation solubility, molecular integrity, adhesion characteristics to human tissue, kinetics of polymerization of the gel including clotting time and clottability, biocompatibility, stability, sterilization, and efficacy tests of the invention made in its preferred method are described in these examples, The examples show that lyophilized fibrin monomer can be rapidly reconstituted with no agglomerates maintaining its molecular integrity.

Homogenized with cross-linked gelatin, the fibrin monomer can form a strong fibrin clot within 1 to 2 minutes of application over a bleeding wound when in contact with the blood. The agent is biocompatible, stable, and adheres to lacerated tissue binding the opposing tissues together. The cross-linked gelatin maintains when homogenized fibrin monomer at an optimal and stable fluid viscosity necessary to deliver the composition up to one hour following preparation.

The following tests were conducted in vitro and in vivo.

### Example 1

### Reconstitution of Lyophilized Fibrin II Monomer and Polymerization of Reconstituted Fibrin II Monomer

### 1.1 Reconstitution Rate

Dissolution times in 2 ml of acetic acid pH 3.5 of 400 mg of lyophilized fibrin monomer-trehalose composition containing 80 mgs of fibrin monomer (40 mg/mL) as described in the invention was compared with lyophilized fibrin monomer without saccharide additive and with fibrin monomer lyophilized only by freeze drying with no previous freeze spray process. In the first formulation 400 mg of lyophilized fibrin monomer-trehalose were filed into a female luer lock syringe of the type Quosina Part C3603, and of 2 ml of acetic acid are filed into male luer lock syringe. In the second and third formulations 80 mg of fibrin monomer were filed into a female luer lock syringe, and of 2 ml of acetic acid are filed into male luer lock syringe. The lyophilized compositions where mixed by means of transferring 10 times forth and back the solubilized mixture from one male luer lock syringe connected to female luer lock syringe 10 times as shown in FIG. 6. The time need to completely dissolve the fibrin powder in the acetic acid is recorded.

The dissolution rate was determined by measuring the concentration of monomer in solution at 20 s, 40 s, 60 s and 120 s in a spectrophotometer at wavelength of 280 nM provides a Vs concentration of fibrin monomer in the solution. The reconstitution rate determined by the amount of fibrin monomer dissolved in the solution is measured by the absorption of light (FIG. 7) 1 mgr of fm equivalent 0.508 optical units at 280 nm

The presence of 200 mg of trehalose per mL of fibrin monomer solution at a concentration of 40 mgrs per mL increases by 2.5 fold the amount of fibrin monomer dissolved within 2 minutes as compared to the two-step lyophilized monomer with no additives and by 15 fold from the single freeze drying step respectively. No visible aggregates were observed in the solution. These results revealed the potential for recovery of native protein using the appropriate reconstitution conditions.

Alternative additives such as sucrose and sucrose-glycine over a range of concentrations were also tested with increases 1.5 fold compared to lyophilized monomer with no additives. When both sucrose and glycine are used at a ratio of 1:1, the activity recovery decreases significantly in activity recovery relative to trehalose in a pattern that is consistent with the inhibition of activity recovery by glycine crystals, despite the presence of an adequate amount of sucrose to afford protection.(14)

### 1.2 Clotting Time

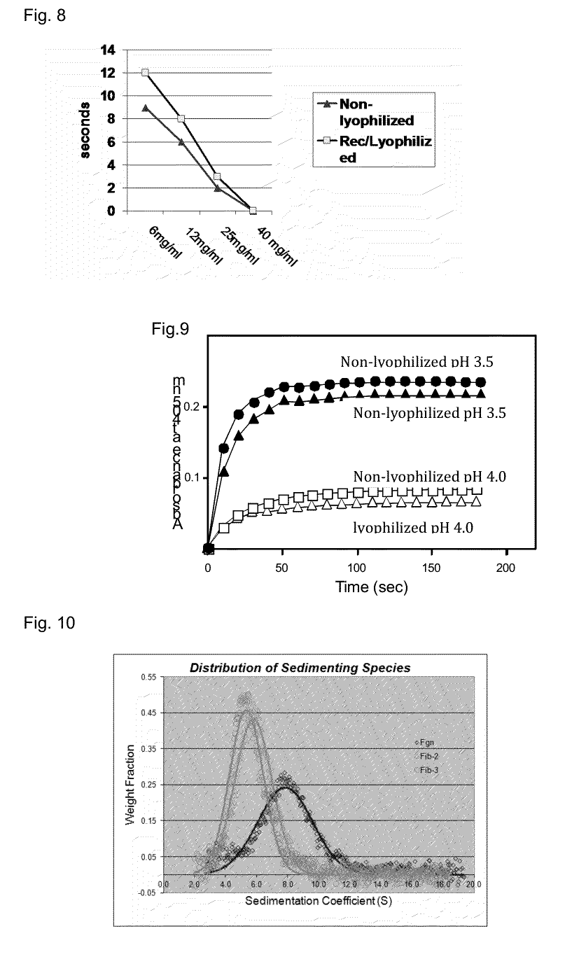

To establish the clotting time of the reconstituted fibrin monomer solution 250 ul of fibrin monomer are mixed with 750 ul of neutralization buffer composed of 50 mM HEPES, 150 mM sodium Chloride and 2.7 mM calcium chloride, which produces a solution of fibrin polymer at a concentration of 12 mg/ml. This concentration is used to record the clotting time because at the 40 mg/ml concentration used by the product, the clotting is so brief that cannot be recorded. At lower concentrations the clotting time is visually recorder with a timer. Three experiments are performed at several concentrations and averaged out (FIG. 8). The average clotting time of reconstituted fibrin monomer when neutralized with buffer at 12 mg/mL was 8 as compared to 6 second before lyophilization. The clotting time delay is attributed to the presence of trehalose in the solution.

### 1.3 Clottability of Reconstituted Fibrin Monomer and Polymerization Measurement by Tubidity

To measure the clottability of the monomer solution, the fibrin polymer formed to determine the clotting time was kept 20 min for maturation, then squeezed to get the possible liquid out, and tested for clottability. Three experiments are performed for each condition and averaged out. The optical density of the liquid is measured in a spectrophotometer and the % Clottability is calculated as follows:

Concentration of fibrin monomer before polymerization−Con. of fibrin monomer in the squeezed liquid*100 Concentration of fibrin monomer before polymerization

The average clottability of reconstituted fibrin monomer was 99.6%

The tuebidity studies that determine the rate at which clot is formed is an additional and more precise manner to assess the polymerization of the fibrin solution,

For turbidity study: Fibrin-monomer samples were diluted to 4.8 mg/mL with solution, pH 3.5 or pH 4.0, at 4° C. 65 μL of fibrin at 4.8 mg/mL at pH 3.5 or pH 4.0 were mixed with 195 μL NB 3.5 or NB 4.0 in 96 well cell culture plate (Cellstar Cat #655180). Fibrin formation was detected at 25° C. by measuring turbidity at 405 nm using ELISA reader. The results presented in FIG. 9 indicate that lyophilized and non-lyophilized fibrins exhibited very similar level of final turbidity at pH 3.5 and 4.0. Final concentration of reconstituted fibrin in fibrin clot was 1 mg/mL. against 1.15 mg/mL in non lyophilized monomer corresponding to a loss of concentration of 15%

### 1.4 Analysis of Sedimentation Profiles

Analytical Ultracentrifugation of lyophilized and non lyophilized fibrin as compared to the fibrinogen used as raw material.

Analysis of the sedimentation profiles of three samples, Fibrinogen used as raw material for production of fibrin monomer and, Fibrin from non-lyophilized and lyophilized monomer shown in FIG. 10 revealed that they all behaved as single species with sedimentation coefficients (S_{w}) equal to ˜8S, ˜6S, and ˜5.5S, respectively, as determined by fitting the radial distribution data with SVEDBERG. No larger species (dimers or higher molecular weight aggregates) were observed in all three samples. Fibrinogen sample Fgn-1 exhibited the expected ˜8S value while both fibrin samples from lyophilized and no lyophilized monomer sedimented slower, most probably due to the primary charge effect, i.e. because charged macromolecules in solvents of low salt concentration display sedimentation coefficients lower than that measured in isoelectric solutions, as described in (15) Analytical Ultracentrifugation experiments confirmed that all three samples, namely, fibrinogen (Fgn-1), non-lyophilized fibrin monomer (Fib-2), and lyophilized fibrin monomer in acetic acid (Fib-3), were monomeric in solution; no significant amount of larger species (dimers, aggregates) were observed (FIG. 10).

### 1.5 Integrity

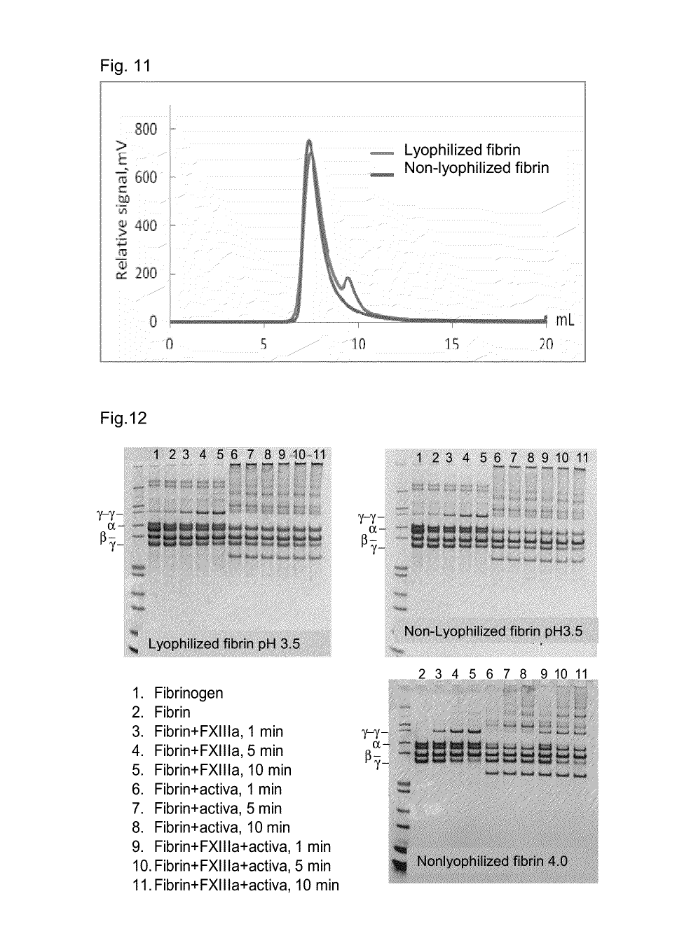

In order to determine the molecular integrity or degradation of the reconstituted fibrin monomer following the lyophilization process as compared to non-lyophilized monomer, HPLC Size exclusion chromatography (SEC) was performed on Agilent 1100 Series HPLC system. Namely, 50 μL samples of non-reconstituted and reconstituted fibrins were applied onto TSKgel G4000SW_{xL} column equilibrated with the same buffer. Flow rate was 1 mL/min and the samples was run in triplicates. The resulted chromatograms are presented on FIG. 11. The elution of both non-lyophilized and lyophilized fibrins began at retention time of 5.52 min, which is a void volume of the column according to the profile of protein standards, and were eluted at the same retention time of 7.48 min. The small pick at 9 m is estimated to correspond to the trehalose component.

### 1.6 SDS-PAGE Analysis of the Non-Lyophilized and Lyophilized Reconstituted Samples

Samples for SDS-PAGE analysis: 20 μL of the non-lyophilized and lyophilized reconstituted samples were mixed with 7 μL of NuPAGE sample buffer (4×). 17 μL of each sample was loaded onto 12 well 4-12% SDS gel. Gels were run at 200V for 40 min than were rinsed with water (10 min) and stained with an Imperial Protein Stain (Thermo Scientific). SDS-PAGE analysis of non-lyophilized and lyophilized fibrin(ogen)s and their cross-linking by activated FXIII and/or Activa at pH 3.5 and 4.0 are presented on FIG. 12. The results indicate that polymerization and cross-linking of non-lyophilized and lyophilized fibrins occurred in the same way at pH 3.5 and 4.0. The cross-linking of lyophilized fibrin is slightly less efficient in comparison to non-lyophilized fibrins due to the trehalose content. The resulted cross-linked products at 5 and 10 min time points seem to form large polymers which cannot enter the PAGE gel.

1.7 Scanning Electron microscopy (SEM): non-lyophilized and lyophilized Fibrin-monomer samples were diluted up to 16 mg/mL (192 μl of each fibrin sample was mixed with 108 μl of the corresponding dilution buffer) to obtain concentration of 16 mg/ml. Then clot formation in the presence of activated FXIII was done by mixing 25 μl of diluted fibrin-monomer with 85 μl of corresponding NB containing 36.4 U/mL of FXIIIa, and the resulted clots were incubated for 1 hour at 37° C. Further preparation of clot samples for SEM were done according to the Ryan protocol (16).

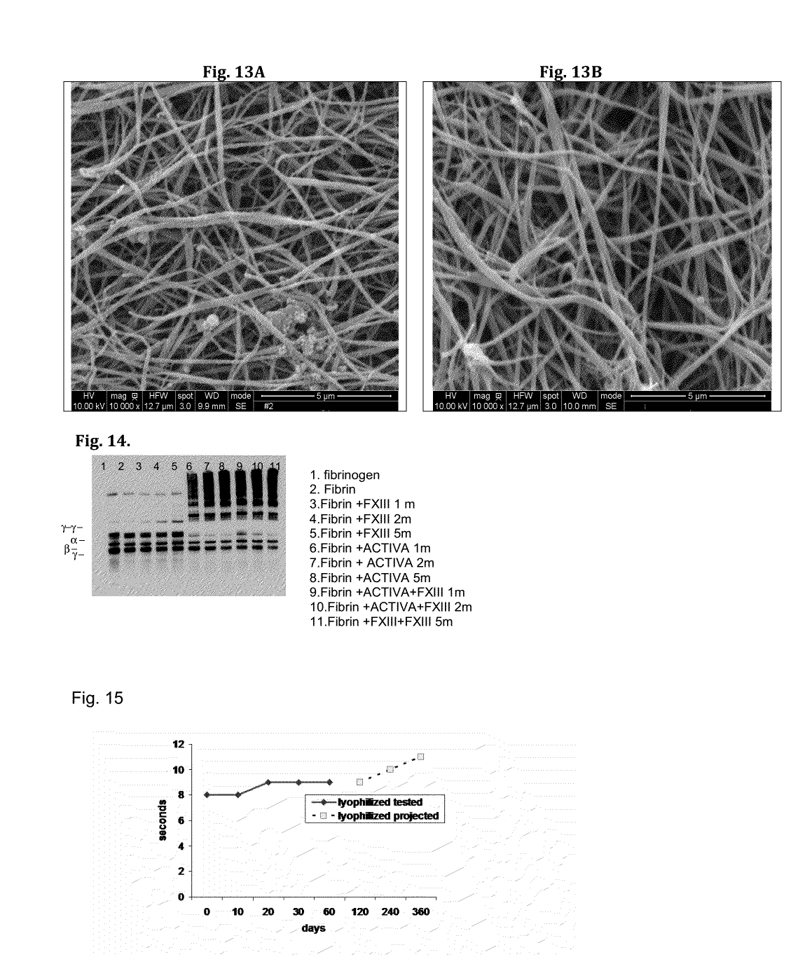

Clots prepared with non-lyophilized fibrin monomer and lyophilized reconstituted fibrin were analyzed by scanning electron microscopy and the resulted SEM images at 10,000 magnifications are presented in FIGS. 13 A and 13B.

The above images suggested similar network morphology in fibrin clot formed by non lyophilized and lyophilized fibrin clots revealed similarity in fiber length and in branch point density.

### Example 2

### Determination of the Effects of Crosslinked Gelatin in the Polymerization and Fluid Viscosity of the Composition

2.1 The polymerization and relative stability of the fluid viscosity, or the resistance of the homogenized composition to flow through a Luer lock syringe of the type Quosina Part C3603 containing 4 mL of the composition was measured by determining the changes in fluid viscosity at 20° C. for 60 minutes at time points 2 m, 5 m, 15 m, 30 m and 60 minutes. These changes were measured determining 1) the resistance to shear stress G′ by rheometry, and the fluidity by measuring the pressure necessary to extrude from the syringe 1 ml per second of the composition; 2) by correlating the changes in pressure necessary to extrude 4 ml of the composition in 1 second from the syringe; and 3) by evaluating visually the polymerization of the homogenized mixture of gelatin in the solution of fibrin monomer by extruding 4 mL of the viscous composition into a petri dish containing 50 mM HEPES and 150 mM sodium Chloride at the following intervals from the time of preparation: 2 m, 5 m, 15 m, 30 m and 60 minutes. Since the polymerization occurs almost instantaneously and therefore the clotting process was recorder visually as Y/N.

TABLE 1
Polymerization and fluid viscosity within one hour of preparation

| | 0 m | 2 m | 5 m | 15 m | 30 m | 60 m |
|---|---|---|---|---|---|---|
| G′ in Pa-s | 130 | 145 | 151 | 155 | 173 | 190 |
| Pressure on piston | 3 psi | 4 psi | 4 psi | 5 Psi | 5 Psi | 7 psi |
| Polymerization | y | Y | y | Y | y | y |

Being 1 cP (center poised)=1 mPa the dynamic viscosity, the aquagel varied between 130,000 cP to 230,000 cP in the one hour with a reduction of fluidity calculated as φ=1/p between 0.00000769 at preparation to 0.00000434 at one hour.

### 2.2 Western Blot Analysis

The polymerization ability of fibrin monomer when homogenized with cross-linked gelatin is established by Western Blot. In order to conduct the study, 22 ul of the homogenized composition is mixed to 22 ul of neutralization buffer containing factor XII, ACTIVA, and Factor XIII plus active. Each composition is incubated for 1, 2, and 5 min. At each time point, the polymerization reaction is stopped with 100 ul of 8M urea, and the sample is diluted with 800 ul of 4M urea. 10 μL of the SDS-PAGE samples were further diluted with 40 μL of NuPAGE sample buffer (1× diluted with H₂O). 10 μL of each sample was loaded onto 12 well 4-12% SDS gel and ran at 200V for 40 min, transferred to the nitrocellulose membrane. iBlot Gel Transfer Stacks nitrocellulose (Invitrogen) were used, Program P3, 8 min. The membrane was blocked with 5% milk in TBS-T for 45 min, then was incubated with 1/5000 diluted anti fibrinogen antibodies-HRP (#SAFG-APHRP 140; 1 mg/mL; Enzyme Research Laboratories) overnight and developed with SuperSignal (Thermo Scientific).

The polymerization process for each sample at each time point is shown in FIG. 14 indicating that fibrin monomer in the homogenized composition formed a cross-linked Clot

### 2.3 Shelf-Life Experiments

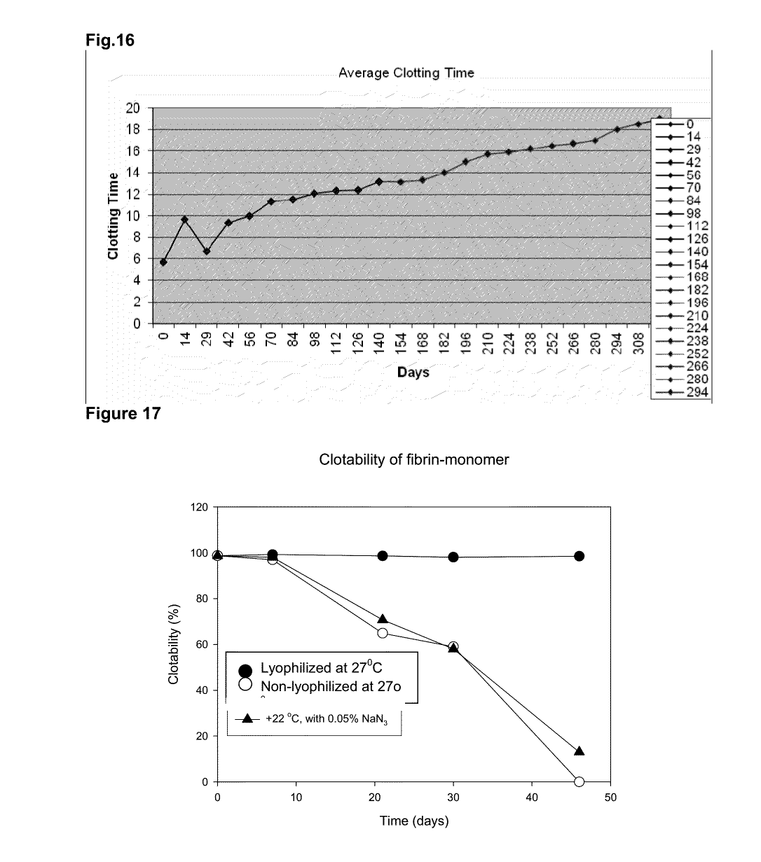

The stability of composition was established by determining the clotting time of the reconstituted fibrin monomer which was stored lyophilized for 90 days (FIG. 15), —The blue line indicates actual testing and red a projection up to 360 days. The experiments establish that lyophilized fibrin monomer does not degrade over time when reconstituted after being stored at room temperature (27° C.). Neither the clotting time nor the fluid viscosity values vary significantly within 90 days of storage at room temperature as compared to the variation of non-lyophilized monomer stored at −20° C. (FIG. 16). Clottability of reconstituted fibrin monomer stored at room temperature was compared to clottability of non-lyophilized fibrin monomer. (FIG. 17)

### 2.3.1 Shelf Life

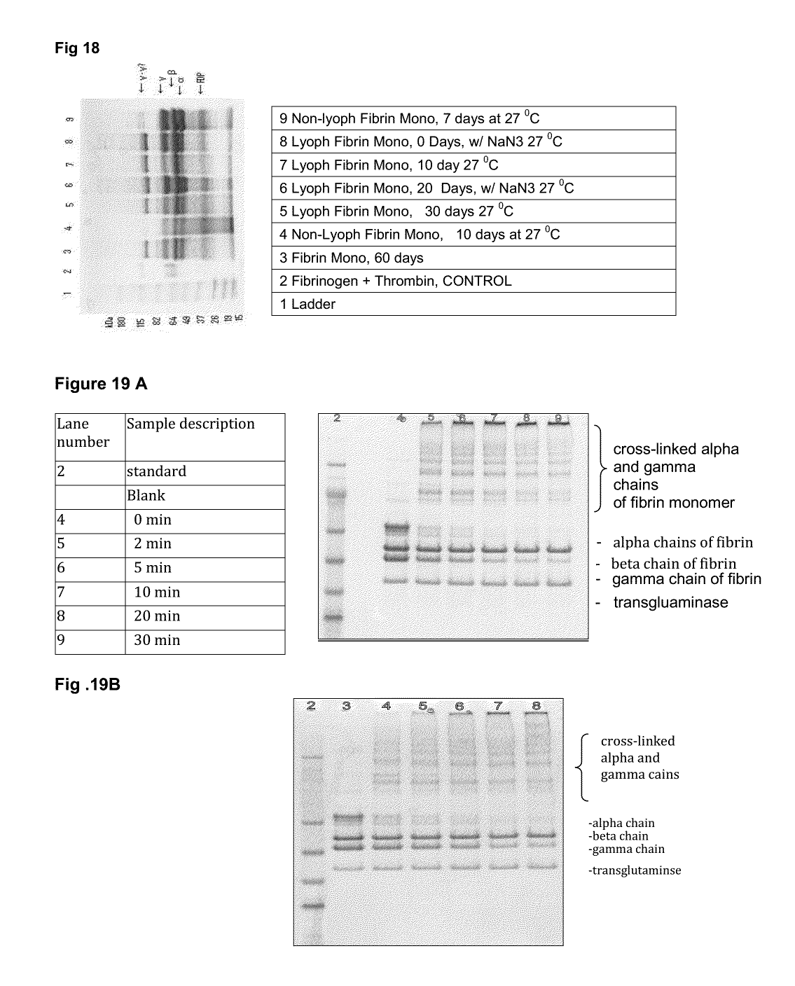

The shelf life of Fibrin monomer once reconstituted was also analyzed by SDS-PAGE. Fibrin monomers isolated by dialysis membrane method were studied in the presence (+/−) of 1% NaN₃, as a preservative. The analyses of these samples were performed at 0,10,20.30 and 60 days upon storage at room temperature; The experiment compared the cross-linking of reconstituted samples at time point 5 minutes following neutralization of the monomer in the presence of factor XIII (FIG. 18). The polymerization and cross-linking in the presence of factor XIII was analyzed when the reaction was stopped at 2, 5, 10, 20 and 30 minutes comparing two samples of fibrin monomer stored at 0 times and at 60 days at room temperature (FIGS. 19A and 19B)

### Example 3

### Studies to Determine the Catalyzing Effect of ACTIVA on Fibrin Stabilization within ClotGel

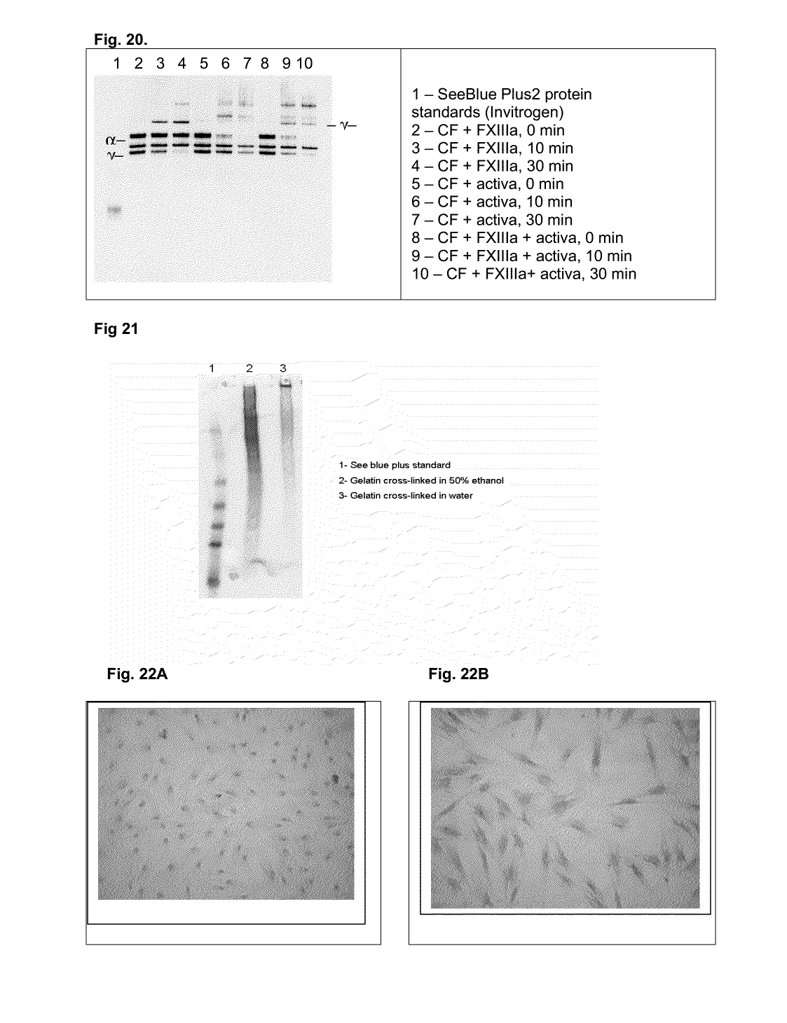

The polymerization of the homogenized mixture was also tested by SDS page. We conducted studies to compare the effectiveness of fibrin monomer polymerization (pH Neutralization) and stabilization (cross-linking) within the homogenized Clotgel mixture by activated Factor XIII versus stabilization by Factor XIII and Ca Independent tranglutaminase enzyme (ACTIVA). It is well established that FXIII in the presence of Ca²⁺ catalyzes fibrin monomer conversion into insoluble fibrin clot. However it was not previously established that there is a synergistic effect of calcium independent transglutaminase enzyme and activated Factor XIII. In order to follow these reactions, fibrin monomer was subjected to calcium independent transglutaminase enzyme treatment, first as a concentration dependent reaction and later as a time dependant reaction. Assays compared a) fibrin and fibrinogen crosslinking by calcium independent transglutaminase enzyme (FIG. 20) and fibrin crosslinking by calcium independent transglutaminase enzyme at concentrations of 20 u/ml, 19 U/ml, and 1 U/ml.

Concentration-dependent and time-dependent monitored reaction (1, 5.10 min, respectively), A volume of acidic solution of 2 mg/ml fibrin was quickly mixed with Activa in 60 mM Tris buffer (pH 8.4, w/2 mM CaCl₂) in variable concentration (1.0-20.0 U/ml) to achieve neutralization. The samples in each lane were incubated for 10 min at 37° C. The samples was electrophoresed and transferred to nitrocellulose membrane. The Fibrin was visualized with anti-fibrinogen antibody (1:50). As expected, FIG. 20 shows that quick neutralization of fibrin with buffer generated a number of cross-linked fibrin molecules (lanes 2, 3, 5) with increased concentration of calcium independent transglutaminase enzyme incorporated in it when compared with lane 1 containing control sample of fibrin. Furthermore, fragmented derivative products (FDP) of lower molecular weight bands also The figure shows the formation of strong gamma dimmers during fibrin cross-linking with calcium independent transglutaminase enzyme and factor XIII at 1 minute. At this time gamma dimmers are not yet present in the fibrinogen sample.

### Example 4

### Crosslinking of Gelatin by Glutaradehyde

The cross-linking of porcine gelatin by glutaraldehyde following the method described above was assessed by SDS-page shown in FIG. 21

### Example 5

### 3. Biocompatibility

ClotGel was tested for biocompatibility with human fibroblasts (HF) as shown in FIG. 22 A and FIG. 22B and human epithelial cells (A549 cell line, ATCC) as shown in FIG. 23 A and FIG. 23B.

Normal human fibroblasts (HFs) were obtained from a commercial source and cultures established in 60 mm tissue culture plates in Dulbecco's modified Eagle's medium supplemented with 10% fetal bovine serum and maintained at 37° C. in a humidified 5% CO₂ atmosphere (CO₂ incubator). Human epithelial cell line A549 was maintained in Minimal Essential Medium supplemented with 10% fetal bovine serum and 2 mM glutamine. When fibroblast and epithelial cell cultures reached subconfluence, control and sodium benzoate ClotGel preparations were placed into individual dishes. The cultures were returned to the CO₂ incubator and examined daily for a total of five days. ClotGel material and medium was removed from all cultures, and adherent cells were stained with crystal violet (0.1% in 2% ethanol).

The main observation was a total absence of damage or toxicity to the cells, and absence of any bacterial or fungal contamination. In human fibroblast cultures exposed to ClotGel preparations, the cells appeared slightly larger or more spread out than in control untreated cultures. Conclusion: ClotGel is biocompatible, and do not affect, but rather stimulate, the growth and differentiation of cells; which is an important attribute in wound healing agents.

### Example 7

### Final Sterilization by Irradiation

The sterility of lyophilized Fibrin Monomer exposed to 30 kGs of gamma radiation was compared to the sterile fibrin monomer solution filtered in a biological safety cabinet using a Nalg-Nunc 500 mL device (Cat #450-0045, nitrocellulose membrane, 0.45 m filter). It must be noted that the dosage does not require the elimination of viruses load because the blood products used as raw materials are USP and FDA approved for human use. The sterilization therefore addresses the possible environmental contamination during the manufacturing process. The lyophilized fibrin monomer (160 mg) filed in a female luer lock syringe of the type Quosina Part C3603 mantained below 27° C. was exposed to a dose of 30 kGs of gamma radiation. Following radiation the lyophilized monomer was tested for sterility and molecular integrity.

### Growth Study:

The general experimental protocol included preparation of sample solutions which were then plated on Potato dextrose agar (PDA, Sigma-Aldrich, Cat#P2182) and Tryptic soy agar (TSA, Sigma-Aldrich, Cat# T4536) gels in Petri dishes for growth. The PDA and TSA gels were incubated and observed at the indicated periods of time for colony growth (mold and/or bacteria) and compared to results obtained by filtered sterilization. The samples were run in duplicate or triplicate with multiple samples indicated with a 1, 2 and 3 designation in data tables. The scale used for evaluation is as follows:

TABLE 2
Colony Count Key

| Symbol | Count |
|---|---|
| − | No visible growth |
| + | 1-199 visible colonies |
| ++ | 200-399 visible colonies |
| +++ | >400 visible colonies |

Table 3 shows the results of studies of microorganism growth analysis on PDA and TSA of the sterile components of FIBRIN_ClotFoam.

TABLE 4
Sterilization Studies by Bacterial Growth on PDA/TSA at 37° C.

| | Potato Dextrose Agar (PDA) | | | | | | | | Tryptic Soy Agar (TSA) | | | | | | | |
|---|---|---|---|---|---|---|---|---|---|---|---|---|---|---|---|---|
| | Time Elapsed (days) | | | | | | | | | | | | | | | |
| Sample | 1 | 2 | 3 | 4 | 5 | 6 | 7 | 11 | 1 | 2 | 3 | 4 | 5 | 6 | 7 | 11 |

| | | | | | | | |
|---|---|---|---|---|---|---|---|
| C* (Fibrin/ | 1 | — | — | — | — | — | — |
| AcOH, | 2 | — | — | — | — | — | — |
| pH 3.5) | 3 | \! | — | — | — | — | — |
| C** (Fibrin/ | 1 | — | — | — | — | — | — |
| AcOH, | 2 | — | — | — | — | — | — |
| pH 3.5) | 3 | — | — | — | — | — | — |

*pre-lyophilized fibrin monomer solution sterilized buy filtration and stored at 4° C. for seven days
**lyophilized fibrin monomer sterilized by gamma ray and reconstituted

The growth data indicate that gamma sterilized fibrin monomer yielded no growth and performed slightly better than filtered composition.

### Example 6

### Use of the Product as an Hemostatic Agent and Sealant

### 6.1. Efficacy in Animal Models

We conducted studies on intracavitary intraoperative bleeding in the swine (pig) model.

### Study Objectives:

Compare ClotGel versus standard surgical practice and Floseal in stopping moderate to severe bleeding in spleen laceration and liver resection

### 6.1.1 Evaluation of ClotGel for the Control of Bleeding as Primary Treatment Following Liver Biopsy

Two groups of Six female Yorkshire crossbred swine, age 2.5 months, weighing 37±2 kg, underwent a 2.5 Inch liver biopsy via open laparotomy, A spot in the middle of the liver was selected to produce the liver injury with a 3 mm biopsy punch. The position was calculated by approximation to the suprahepatic vessels and some branches of the portal vein. The spot was marked with a marker. In Group 1 the resection treated with a 1 mL ClotGel and compressed for 2 minutes over the wound. In group 2 (n=6) the same resection was treated with 1 ml of Floseal and compressed for 2 minutes against the wound.

### Results:

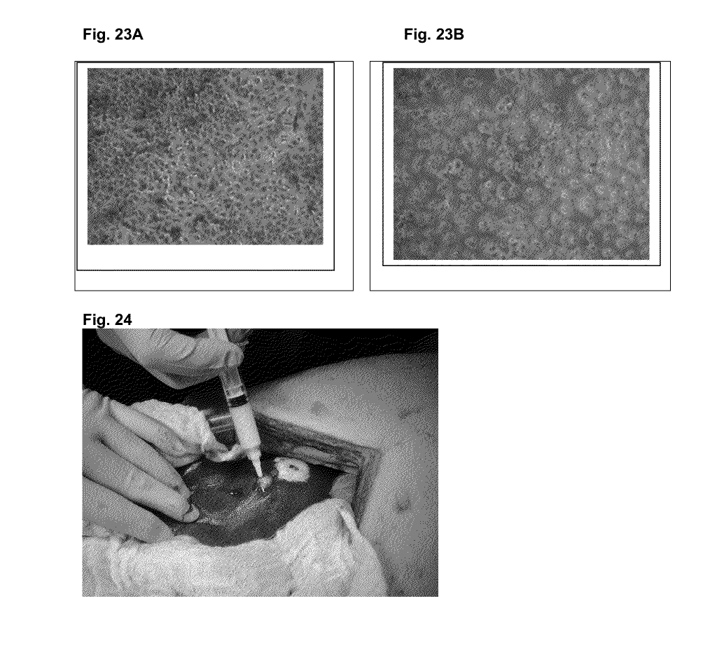

In both groups hemostasis was achieved in all animals within 2 minutes of application. (FIG. 24)

### 6.1.2 Evaluation of ClotGel for the Control of Bleeding as Primary Treatment Following Partial Nephrectomy

The purpose of this study is to determine if CloGel can stop profuse bleeding within 5 minutes of application in cases of partial kidney resection.

### Methods:

Four female Yorkshire crossbred swine, age 2.5 months, weighing 37±2 kg, were used. The protocol was approved by the Institutional Animal Care and Use Committee.

Animals were subject to a 1.5 inch thick resection of the lower portion (created sharply by an 11 blade scalpel). After the damage was induced, 3 mL of ClotGel composition was compressed against the laceration for 2 minutes. Hemostasis was achieved in all animals within 2 minutes of application. None of animals treated with 3 mL of Floseal (Baxter) achieved complete hemostasis.

### 6.1.3. Evaluation of ClotGel for the Control of Bleeding as Primary Treatment in Spleen Laceration.

The purpose of this study is to determine if CloGel can stop profuse bleeding within 5 minutes of application in cases traumatic spleen laceration.

### Methods:

Four female Yorkshire crossbred swine, age 2.5 months, weighing 37±2 kg, were used. The protocol was approved by the Institutional Animal Care and Use Committee.

Animals (N=4) were subject to a 1 inch incision in lateral middle portion of the spleen (created sharply by an 11 blade scalpel). After the damage was induced, 3 mL of ClotGel composition was compressed against the laceration for 2 minutes. Hemostasis was achieved in all animals within 2 minutes of application*. None of animals treated with 3 mL of Floseal (Baxter) achieved complete hemostasis.

*A five minute time to hemostasis is defined by the Blood Products Committee of the Food and Drug Administration as the maximum time to demonstrate efficacy in achieving hemostasis.

### Example 7

### Pharmacokinetic Profile of the Agent Through Biodegradation Studies

Elimination through biodegration by proteolytic enzymes was determined in vivo.

### Method:

To examine the fate of Clotgel in vivo, a batch was prepared using fluorescein-tagged human fibrinogen as tracer. This preparation of ClotGel was applied to the four animals of Group 1 in the liver biopsy group. Animals were euthnanized at 2 weeks (n=2) and 4 weeks (n=2) following application Organs were collected, fixed in 10% formalin and embedded in paraffin blocks. Histologic sections were examined at 100× and 400× in fluorescence microscope. The elimination of ClotGel was determined by either the total absence of fluorescent traces in the samples, or by the level of fluorescense observed at 2 weeks and 4 weeks.

### Results:

ClotGel was eliminated in all organs within 4 weeks of application.

What is claimed is:

1. A composition for the control of bleeding in humans with or without compression comprising:
a) lyophilized desAB fibrin monomer (fibrin II)
b) acetic acid solution for reconstitution of lyophilized fibrin monomer
c) cross-linked gelatin

2. The composition as claimed in claim 1 wherein the acetic acid solution has a pH of 3.4-3.5.

3. The composition as claimed in claim 1 wherein the fibrin monomer in acid solution is mixed 5:1 with dihydrate trehalose USP-NF previous to lyophilization.

4. The composition as claimed in claim 1 wherein the mixture of fibrin monomer with trehalose is lyophilized in particles of 50 μm to 200 μm containing at least 20% fibrin II monomer.

5. The composition as claimed in claim 4 wherein the lyophilized fibrin monomer can be reconstituted in less than two minutes by dissolving the fibrin microparticles it in a solution of acetic acid at pH 3.5

6. the composition as claimed in claim 4 wherein the fibrin monomer can be reconstituted in acetic acid solution at a concentration ranging from 20 mg/mL to 40 mg/ml.

7. The composition as claimed in claim 4 wherein the lyophilized fibrin monomer maintains the polymerization and cross-linking properties of non-lyophilized fibrin monomer in acid solution.

8. The composition as claimed in claim 4 wherein the lyophilized fibrin monomer is stable at room temperature

9. The composition as claimed in claim 1 wherein the gelatin is of porcine or bovine origin.

10. The composition as claimed in claim 1 wherein the gelatin is cross-linked with glutaraldehyde.

11. The composition as claimed in claim 8 wherein 10 gr. of gelatin is dissolved in 100 mL of 50% water and 50% ethanol

12. The composition as claimed in claim 8 wherein 0.2% of glutaraldehyde is added to a solution of gelatin in water and ethanol.

13. The composition as claimed in claim 8 wherein the glutaraldehyde is inactivated by 0.1% of borohydrate or 1% to 5% of glycine.

14. The composition as claimed in claim 11 wherein the crosslinked gelatin is washed free of inactivating agent with 100% ethanol.

15. The composition as claimed in claim 12 wherein the cross-linked gelatin is dried in an incubator at 37° C. overnight

16. The composition as claimed in claim 1 wherein the reconstituted fibrin monomer is homogenized with the cross-linked gelatin to be applied over the bleeding wound.

17. The composition as claimed in claim 1 wherein the fibrin is free of thrombin.

18. The composition as claimed in claim 1 wherein the homogenized like gel that is formed is characterized as being biocompatible and biodegradable for human use.

19. The composition as claimed in claim 1 wherein the components can be sterilized by gamma radiation

20. The composition as claimed in claim 1 n wherein it can be stored at room temperature

21. The composition as claimed in claim 1, when applied and becoming in contact with blood over the wounded tissue effects the formation of a covalently bonded fibrin clot formed by long fibrin fibers that seals the wound.

22. The composition as claimed in claim 18 wherein the fibrin clot is biodegradable.

23. A composition as claimed in claim 18 wherein hemostasis is effected within two minutes of application of the composition to the wounded tissue, with or without compression.

24. A method for preparing a composition for the control of bleeding, with or without compression, comprising the steps of:
a. Preparing a desAB fibrin (fibrin II) monomer in acetic acid solution at a concentration ranging from 6 mg/mL to 40 mg/mL.
b. Lyophilizing the fibrin monomer solution in acetic acid by a two-step process consisting of: freeze granulation in liquid nitrogen followed by freeze drying of granulated particles.
c. Cross-linking gelatin in the presence of a cross-linking agent as to obtain a non-aggregating granulated gelatin when it is dried, wherein the gelatin is cross-linked with 0.2% of glutaraldehyde. in a solution of 50% water, and the crosslinking is inactivated by 0.1% of borohydrate or 1% to 5% of glycine, the crosslinked gelatin is washed free of inactivating agent with 100% ethanol, gelatin is dried in an incubator at 37° C. overnight the crosslinking agent comprises maintains a stable viscosity when homogenized with fibrin monomer in solution.
d. Reconstituting the lyophilized fibrin monomer into an acetic acid solution at pH 3.5
e. homogenizing the fibrin II monomer in solution with the cross-linked gelatin as to obtain a viscous gel-like or aquagel composition.

25. The method as claimed in claim 21, wherein the lyophilized fibrin II monomer is obtained by
a. freeze spraying fibrin monomer solution in acetic acid into spherical particles of 50 μm to 100 μm in liquid nitrogen
b. lyohilizing the frozen granulated frozen fibrin monomer in a freeze drying equipment.

21. The method as claimed in claim 18 wherein porcine gelatin solution in 50% water and 50% ethanol is cross-linked by 0.2% glutaraldehyde and incubated at room temperature for three hours at pH 8.0 to 9.0.

22. The method as claimed in claim 18 wherein the cross-linking reaction is stopped by 0.1% of borohydrate or 1% to 5% of glycine.

23. The method as claimed in claim 18 wherein the inactivated cross-linked gelatin is washed free of inactivating agent with 100% ethanol.

24. The method as claimed in claim 18 wherein the crosslinked gelatin is dried in an incubator at 37° C. overnight.

25. A method as claimed in claim 18 wherein the lyophilized Fibrin II monomer is reconstituted in a solution of acetic acid pH 3.5 by gently mixing the fibrin powder in the acetic acid solution for 2 minutes.

26. A method as claimed in claim 18 wherein the cross-linked gelatin is homogenized with reconstituted fibrin II monomer by means of male luer lock syringe connected to a females luer lock syringe that allows the composition to be extruded forth and back between the syringes.
